(19) United States
(12) Patent Application Publication
(10) Pub. No.: US 2025/0235386 A1
(43) Pub. Date: Jul. 24, 2025

(54) AUTOMATED PILL FULFILLMENT SYSTEMS AND METHODS

(71) Applicant: CVS Pharmacy, Inc., Woonsocket, RI (US)

(72) Inventors: Michael Joseph Szesko, Freehold, NJ (US); Dennis Wayne Rice, Flanders, NJ (US); James Jon Lacomis, Bear Creek Township, PA (US); Stephen Webb, Hickory Creek, TX (US); Thomas Purvis, Gainesville, FL (US); Chih-Jen Leu, East Brunswick, NJ (US); Joseph T. DeDeo, Mount Laurel, NJ (US); William David Cochrane, South Abington Township, PA (US); Stephen John Wilson, Cambridge (CA); Evan Andrew Walsh, Renfrew (CA)

(21) Appl. No.: 19/030,136

(22) Filed: Jan. 17, 2025

Related U.S. Application Data
(60) Provisional application No. 63/622,360, filed on Jan. 18, 2024.

(51) Int. Cl. A61J 7/00; A61J 7/02; G16H 20/13

(52) U.S. Cl. CPC A61J 7/0076; A61J 7/02; G16H 20/13; A61J 2200/70; A61J 2205/10; A61J 2205/40; A61J 2205/60

(57) ABSTRACT

An automated pill order filling system includes a main conveyor and track running from a first end to a second end of the main conveyor. A plurality of pucks, each configured to hold a respective pill container, travel on the track. The system includes a first array of automatic pill dispensers, configured to dispense a first number of pills into a first designated pill container at a first fill rate. The system also includes a second array of automatic pill dispensers, disposed adjacent the first array of automatic pill dispensers, configured to dispense a second number of pills into a second designated pill container at a second fill rate that is lower than the first rate. The first and second arrays of automatic pill dispensers are configured to operate independently of one another.

### CROSS-REFERENCE TO RELATED APPLICATIONS

The present application claims the benefit of and priority, under 35 U.S.C. § 119 (e), to U.S. Provisional Application Ser. No. 63/622,360, filed on Jan. 18, 2024, entitled “AUTOMATED PILL FULFILLMENT SYSTEMS AND METHODS,” the entire disclosure of which is hereby incorporated herein by reference, in its entirety, for all that it teaches and for all purposes.

### BACKGROUND

The present disclosure is generally directed to automated systems and, in particular, toward a combined multiple-rate automated pill fulfillment system and method.

Conventional pill counting and filling operations typically require careful monitoring, validation, and/or verification by a qualified pharmacist. In particular, pharmacists are generally needed to ensure that the appropriate number of pills, the correct type of pill, and absence of any desiccants or foreign matter, of a specific prescription are compiled for a properly identified patient, or customer. As can be appreciated, errors in any one or more of the count of the pills, the selection of the type of prescription to be filled, or of the end user (e.g., the patient) of the pills can cause unintended health issues, if not fatalities. Accordingly, the accuracy of fulfillment is critical to the health of the end users and to ensuring reliable and trusted operations.

A single pharmacist is generally only capable of accurately fulfilling a limited number of prescriptions per day. While automatic pill counters may be used to assist in ensuring, within an accepted deviation, a number of pills in a container, a pharmacist is still required to inspect the type of pill counted and cross-reference the prescription information associated with a pill order to an appropriate patient. Since these operations are highly dependent on the capabilities of a pharmacist, increasing the output of a conventional pill fulfillment center necessarily includes increasing the number of pharmacists who are available to process orders.

### BRIEF SUMMARY

It is with respect to the above issues and other problems that the examples presented herein were contemplated. The present disclosure provides automated pill fulfillment systems and methods that are capable of accurately and simultaneously processing prescription filling at variable rates, without requiring verification by a pharmacist. In some examples, the automated pill fulfillment systems and methods described herein may not require a pharmacist to remotely access and view images of pill container, or vial, contents (e.g., taken during fulfillment) and compare these images to expected national drug code (“NDC”) directory drug images on a computer screen. For instance, in the automated pill fulfillment systems and methods described herein, a pharmacist may review the replenishment container for one or more automated counting cells, and may not be required to view the images of the pill container, or vial, contents. In some examples, the accuracy and repeatability of the automated pill fulfillment systems and methods may be verified by a Board of Pharmacy that accepts this type of validation testing process in lieu of requiring a pharmacist perform image compares of 100% for all of the prescription bottles. A pharmacist may still be involved in the process, even if only to provide oversight of the overall process. This pharmacist may be referred to herein as the pharmacist in charge (“PIC”) and whose name may be on the prescription. The automated pill fulfillment systems and methods described herein are capable of preventing errors attributed to conventional pill counting machines by providing, among other things, advanced vision technology configured with high-speed pill color and shape recognition and positive transfer technology that prevents stray or erratic pill dispenses. In some examples, one or more quality assurance and exception checking stations may be included to verify and validate the accuracy of dispenses for a number of pill dispensers. A recorded image may be taken of each pill as it is being dispensed. This recorded image may be provided for system validation, station validation, and/or to verify the proper dispense if there is any question of accuracy in type of pill or pill count.

In some examples, the automated pill fulfillment system (“APFS”) may include a main conveyor that is capable of transferring one or more pill containers along a track from a labeling station, to at least one pill dispenser in an array of pill dispensers, and then to a capping station before the capped pill container is packaged for shipping to an end user (e.g., a patient, customer, etc.) according to a pill order. In some examples, an image capture station may be located prior to the capping station. This image capture station may capture the pill image inside the pill container, or vial. These captured images may be used in a process where a pharmacist inspects the contents of one or more pill containers, or vials. In one example, artificial intelligence (“AI”) may be employed (e.g., in lieu of a pharmacist) to provide an automated system that is capable of performing image comparisons and validations that the correct drug is present in the correct pill container, or vial. The pill order may define a specific number of pills that are associated with a specific pill type for an end user. In some examples, the pill order may include pharmacy information, mailing information, warnings, patient name, routing information, etc., combinations thereof, and/or other information. This information may be obfuscated, secured, cryptographically protected, or otherwise prevented from detection.

As described herein a “pill” may be used to refer to one or more of a chewable, capsule, spansule, softgel, liquid gel, tablet, solid medication, suppository, vitamin, drug, etc., and/or combinations thereof.

The labeling station may automatically determine that a pill container is present at an application area and, in response, apply a label onto the outside of the pill container. The label may correspond to a flat material substrate (e.g., paper, polyester, vinyl, etc.) with an adhesive layer disposed on an attachment side thereof. As the adhesive layer contacts the outside surface of the pill container, the label may transfer from the labeling station and be applied to the pill container. The label may include information printed there or otherwise encoded as part of the label (e.g., including an embedded radio frequency identification tag, memory storage device, or other circuit and/or chip, etc.). The information may include, but is in no way limited to, a pill order, an identification of the prescription, end user, count of pills required, dosage amounts, usage instructions, and/or other routing information used by the APFS. In one example, the label may include a barcode (or multiple barcodes that enable the verification of that barcode downstream at the image capture station and/or the packaging station, etc.) that includes some or all of this information. The barcode may be any type of barcode or machine-readable code (e.g., quick response (“QR”) code, data matrix two-dimensional code, radio frequency identification (“RFID”) tag or label, code 39 one-dimensional alphanumeric code, code 128 high-density linear barcode, interleaved 2 of 5 numeric code, stacked linear two-dimensional barcode, etc., and/or combination thereof) printed to a visible side of the flat material substrate of the label.

In one example, the pill container may be physically placed into, or onto, a material transfer “puck.” The puck may correspond to a machined block, pallet, or other device that is capable of positively holding the pill container at least during transfer operations by the main conveyor. In one example, the main conveyor may correspond to a shuttle transport system. In some examples, the puck may selectively engage with a shuttle that travels along the track of the main conveyor. The puck may selectively engage with one or more spurs with diverts off from the main conveyor (e.g., for pill dispensing, capping, packaging, etc.). For instance, the track of the main conveyor may be configured as a loop extending from a first point (e.g., a first end of the conveyor) in a first direction along a first side to a second point (e.g., a second end of the conveyor), and then extending from the second point in a second direction (e.g., opposite the first direction) along a second side (e.g., opposite the first side) back to the first point. The main conveyor may include a plurality of shuttles that are configured to travel around the loop as part of the main conveyor. In one example, the plurality of shuttles may travel around the loop independently from one another. In any event, the loop arrangement allows the shuttles to continuously move, or repeatedly cycle, around the track.

Each shuttle may include a receiving area (e.g., a slot, opening, cavity, etc.) that is capable of receiving and/or capturing a puck. In some examples, a puck (e.g., holding a pill container, etc.) may be indexed into a shuttle for traveling along the track. The puck may magnetically interface with the shuttle (e.g., by a magnet positioned between the puck and the shuttle, or vice versa) or the puck may otherwise engage with the shuttle such that the puck is prevented from disengaging from the shuttle during transfer. Once engaged, the shuttle may be conveyed to one or more stations positioned along the track of the main conveyor. In one example, the puck may be selectively disengaged from the shuttle to be processed by one or more stations along the track of the main conveyor or a spur (e.g., for capping, packaging, and/or performing some other function). In this example, the shuttle may be allowed to continue to convey along the track of the main conveyor or remain in place until the puck returns to the shuttle (e.g., after an operation is performed, etc.). In the event that the shuttle is allowed to continue along the track of the main conveyor, the puck may be engaged with a different shuttle of the main conveyor for further processing. By way of example, a puck may be disengaged from a shuttle at a first station where a first operation (e.g., pill filling, etc.) is to be performed and the shuttle may then be conveyed away from the first station (e.g., to pick up another puck, engage with a different puck, etc.). When the first operation is completed, the puck may be engaged with another (e.g., the same or different) shuttle that is directed (e.g., by a controller, etc.) to receive the puck at the first station. Among other things, this arrangement allows for the continuous movement of shuttles along the track of the main conveyor without slowing the throughput or processing of other pucks and operations.

Based on information associated with pill order, the pucks (e.g., within the shuttle) may be conveyed along the track to a select automatic pill dispenser in the APFS. In some examples, the APFS may include one or more barcode scanners that are configured to read the barcode on the label of a pill container as it is conveyed throughout the APFS. Examples of locations where a barcode may be read include, but are in no way limited to, a point at the label printer (e.g., to verify the correct and complete label is included on the pill container, etc.), a point at the pill image capture station just prior to capping (e.g., to verify the pill container contents, etc.), and a point just prior to the insertion of the bottles and printed documents into the bag during the packing operation (e.g., to confirm the correct bottles & printed documents are included, etc.). In some examples, the information contained in the barcode may determine which automatic pill dispenser is needed to fill the pill order and the shuttle may be automatically directed to that determined automatic pill dispenser. The automatic pill dispenser may be part of an array of automatic pill dispensers. The automatic pill dispenser may be determined based on one or more of a type of pill to be dispensed, an amount of a pill to be dispensed, and/or a time for fulfillment for the pill order. In some examples, the automatic pill dispensers may be arranged in groups, or arrays, based on a fill rate associated with each automatic pill dispenser. For instance, a first array of automatic pill dispensers may be disposed along a length of the track between the first point and the second point. At least some of the automatic pill dispensers in the first array of automatic pill dispensers may be configured to dispense a number of pills into a pill container at a first rate (e.g., a high-volume flow rate, etc.). The rate may correspond to an order popularity associated with pills dispensed by the automatic pill dispensers. For example, the first rate may be associated with pills that are more commonly, or frequently, prescribed than pills associated with the second rate. Pills that are more commonly prescribed than others require routine and frequent replenishment. Moreover, the first rate automatic pill dispensers that dispense these types of pills may need to operate more quickly and/or with higher throughputs than the second rate automatic pill dispensers that are associated with less commonly prescribed pill types.

The automatic pill dispensers described herein may be configured to use vision to determine shape and color recognition for pill counting. In some examples, this vision technology may count multiple pills at the same time. The vision counting provided by these automatic pill dispensers may be impervious to dust (e.g., since the camera is located well outside of the pill dispense, or drop, path). The vision system may be able to detect color and shape variation in the event a stray, or nonconforming, pill may be included in the dispenser. Not only is the vision system of the automatic pill dispensers configured to confirm the correct pill color and shape is dispensed, but the vision technology can record and store a small video file of every pill dispensed into a pill container. This data may be used to validate machine capabilities, determine dispensing accuracy, track dispenses made over time, provide traceability of dispenses associated with pill orders, and guarantee the quality of a dispense made, to name a few.

In some examples, the first rate automatic pill dispensers may be arranged along the track of the main conveyor and/or one of several spurs that have divert sections to the main conveyor (e.g., providing fast automated filling that is close to the main conveyor) and the second rate automatic pill dispensers may be arranged apart from the track of the main conveyor (e.g., providing selective low volume filling that is located a distance away from the main conveyor but that may still be connected to the spur of the main conveyor). In one example, where the pill order defines that a particular pill is to be dispensed from the second rate automatic pill dispensers, the APFS may instruct a robot (e.g., gantry robot, six-axis robot, pick-and-place system, etc.) to remove a pill container from a puck, which is in a stationary position in the station, from the main conveyor and transfer the pill container to a separate second rate automatic pill dispenser in an array of separate second rate automatic pill dispensers. The array of separate second rate automatic pill dispensers may operate at a lower rate of speed than the first rate automatic pill dispensers. Once the pill container is filled with the appropriate number of pills from the select second rate automatic pill dispenser, the robot may be instructed by the APFS to return the filled pill container to the main conveyor by, for example, by engaging the filled pill container with a puck and shuttle on the main conveyor. The filled pill container and/or puck may then be conveyed by the APFS to an image capture station, a capping station, a consolidation station, a quality assurance or exception handling station, and/or a packaging station.

The capping station may include a capping machine that is configured to seal and/or cap the filled pill container. In some examples, the capping machine may include a protective seal application machine that applies and seals (e.g., heat seals, etc.) a foil, or other material, to cover an opening of the filled pill container. A desiccant, cotton ball, or other insert may be placed into the filled pill container prior to sealing and/or capping. In any event, the capping machine may apply a cap to the opening of the filled pill container. The cap may correspond to clip-on cap or a screw-on cap. The cap may be configured to be childproof and/or tamperproof. The cap may be configured to be senior-friendly, arthritis-friendly, and/or include features that aid opening by those with injuries, disabilities, and/or limited tactile abilities.

In some examples, the APFS may include a quality control, or quality assurance and exception (“QA”), station. The QA station may routinely and/or randomly check the accuracy of a dispense made by one or more of the automatic pill dispensers in the APFS. The QA station may be used to validate an automatic pill dispenser in the APFS as providing zero failures over a predetermined number of dispenses. A validated automatic pill dispenser may be exempted from one or more future QA checks. The QA station may include a vision system (e.g., camera, etc.) and one or more detectors that are capable of verifying a pill type and a number of pills in a filled pill container. The QA station may employ image recognition that is capable of detecting a color, shape, and/or marking (e.g., decoration, text, image, etc.) of pills. In some cases, the images taken at the QA station may be sent (e.g., across a wireless communication network, etc.) to a certified or otherwise qualified pharmacist for review, rejection, verification, or approval. The verification provided by the pharmacist may be used to qualify a particular automatic pill dispenser in one or more arrays of automatic pill dispensers.

Once the pill containers have been filled with the specific number and type of pill, the image capture station (e.g., including one or more cameras or image sensors, etc.) may take an image of the contents of the pill containers and then the filled pill containers are capped. The filled and capped pill containers may then be conveyed to a packaging station for preparing to send the same to a client and/or recipient. The clients and/or recipients may include, but are in no way limited to, an end user (e.g., a patient, etc.), a pharmacy, a pharmacist, a hospital, a doctor, a clinic, a care center, a healthcare professional, a distribution center, and/or some other entity and/or location. The packaging station may include a bagging system that is capable of receiving filled and capped pill containers in a shipping container such as a bag, box, envelope, and/or the like. The packaging station may include at least one printer that is configured to print instructions, medication details, prescription information, and/or the like onto an insert sheet. In some examples, the packaging station may include one or more barcode readers to validate the correct pill containers and printed documents are to be inserted into the shipping container (e.g., bag, etc.). The packaging station may include one or more pick-and-place systems, or robots, that are capable of inserting the insert sheet into the shipping container along with the filled and capped pill containers. In some examples, the shipping container may be configured to receive multiple filled and capped pill containers that are associated with one or more pill orders. In some examples, the shipping container may be labeled, or printed, with shipping information for the client or recipient associated with the pill order. The shipping information may include a destination address, a source address, recipient identification (e.g., patient name, etc.), source identification, and/or postage information (e.g., metered postage, etc.).

All examples and features mentioned above can be combined in any technically possible way.

The preceding is a simplified summary of the disclosure to provide an understanding of some aspects of the disclosure. This summary is neither an extensive nor exhaustive overview of the disclosure and its various aspects, examples, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, examples, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, examples, and configurations of the disclosure, as illustrated by the drawings referenced below.

FIG. 1A shows a first schematic plan view of an automated pill fulfillment system in accordance with examples of the present disclosure;

FIG. 1B shows a second schematic plan view of the automated pill fulfillment system in accordance with examples of the present disclosure;

FIG. 1C shows a block diagram of the automated pill fulfillment system in accordance with examples of the present disclosure;

FIG. 2 shows a perspective view of multiple arrays of automatic pill dispensers in accordance with examples of the present disclosure;

FIG. 3 shows a perspective view of an array of automatic pill dispensers in accordance with examples of the present disclosure;

FIG. 4A shows a perspective view of a pill container shuttle of a labeling conveyor holding a first size of pill container in accordance with examples of the present disclosure;

FIG. 4B shows a perspective view of the pill container shuttle of the labeling conveyor holding a second size of pill container in accordance with examples of the present disclosure;

FIG. 5A shows a perspective view of main conveyor shuttle holding a pill container in a material handling puck according to examples of the present disclosure;

FIG. 5B shows a section top plan view taken along line 5B-5B of the pill container and material handling puck shown in FIG. 5A in accordance with examples of the present disclosure;

FIG. 5C shows a side elevation view comparing a fill height for a pill container shuttle with a first puck and pill container size and a second puck and pill container size in accordance with examples of the present disclosure; and

FIG. 6 is a flow diagram of a method for automatically filling containers using the automated order fulfillment system in accordance with examples of the present disclosure.

### DETAILED DESCRIPTION

Before any examples of the disclosure are explained in detail, it is to be understood that the disclosure is not limited in its application to the details of construction and the arrangement of components set forth in the following description or illustrated in the drawings. The disclosure is capable of other examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides examples only, and is not intended to limit the scope, applicability, or configuration of the claims. Rather, the ensuing description will provide those skilled in the art with an enabling description for implementing the described examples. It being understood that various changes may be made in the function and arrangement of elements without departing from the spirit and scope of the appended claims.

Various aspects of the present disclosure will be described herein with reference to drawings that may be schematic illustrations of idealized configurations.

Conventional pill order fulfillment generally requires pill counting and filling by a qualified pharmacist. As can be appreciated, a single pharmacist is usually only capable of accurately fulfilling a limited number of prescriptions per day. In an effort to increase production, efforts have been made to utilize sensor-based automatic pill counters. These sensor-based automatic pill counters are typically only able to detect a presence or absence of a pill. However, the sensor-based automatic pill counters are unable to determine a shape, color, or other features associated with pills to be dispensed. Further, many pills that are dispensed using these sensor-based automatic pill counters are dispensed into a staging area before being dropped into a pill container, or vial. This process is slow and may result in inaccurate counts, spilled pills, or an accumulation of dust in the system. Moreover, these conventional sensor-based automatic pill counters are susceptible to failure due to the accumulation of dust, small pill parts, or other detritus blocking a sensing area of the counting sensor used in the sensor-based automatic pill counters. As can be appreciated, a pharmacist is still required to inspect the type of pill counted and cross-reference the prescription information associated with a pill order to an appropriate patient. Since these operations are highly dependent on the capabilities of a pharmacist, increasing the output of a conventional pill fulfillment center necessarily includes increasing the number of pharmacists who are available to process orders.

It is with respect to the above issues and other problems that the examples presented herein were contemplated.

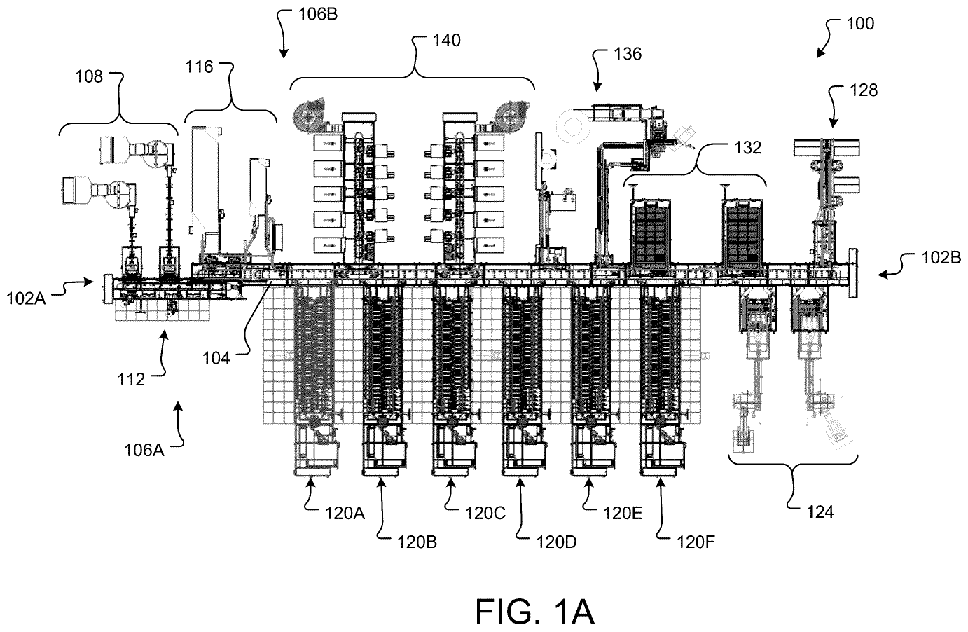

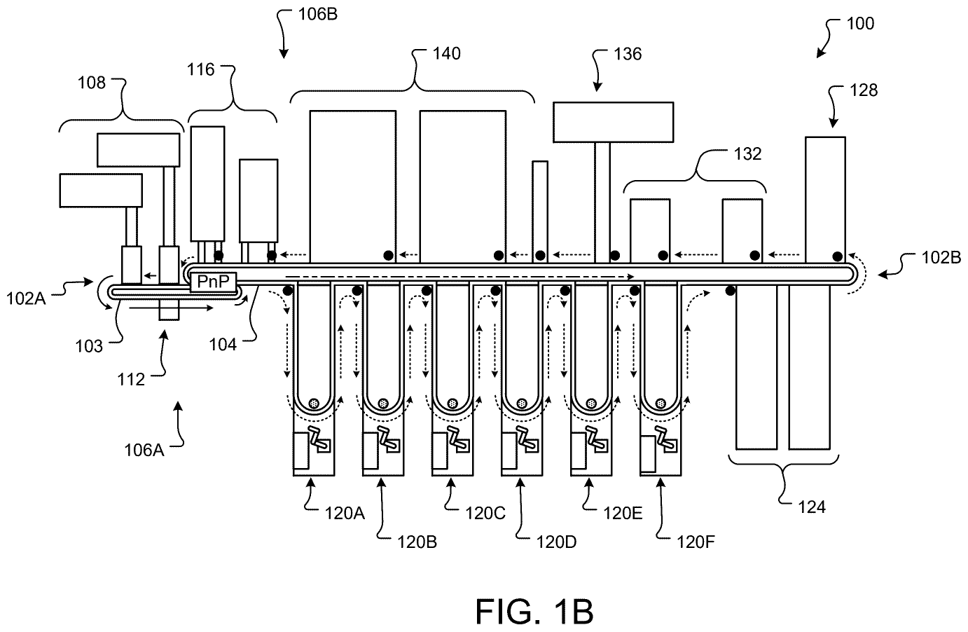

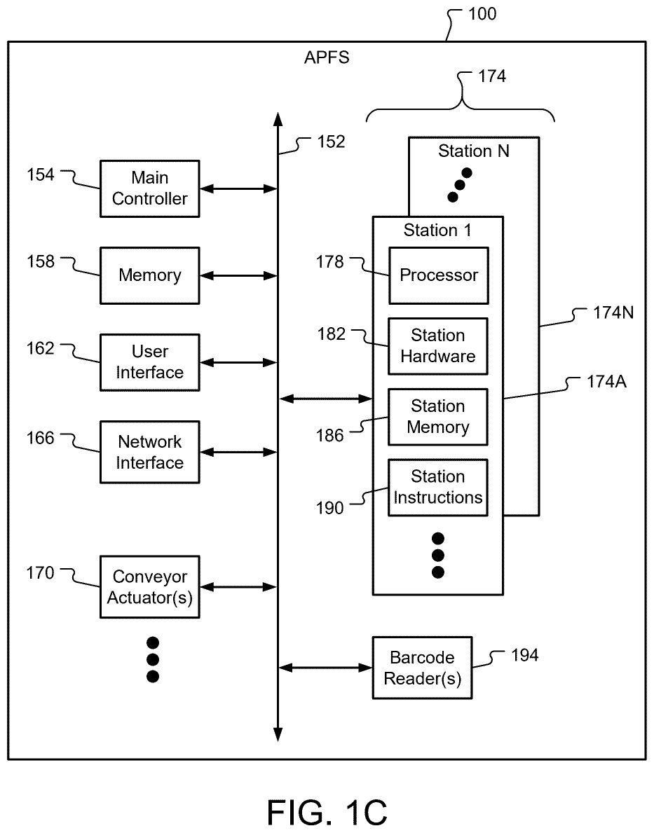

Referring now to FIG. 1A, a first schematic plan view of an automated pill fulfillment system (“APFS”) 100 is shown in accordance with examples of the present disclosure. The APFS 100 may be arranged as a group of integrated systems that are linked together by one or more material handling and transfer units, or conveyors (e.g., pallet power-and-free conveyor, walking beams, flexible link conveyor, belt conveyor, roller conveyor, independent shuttle control track conveyor system, etc.). As illustrated in FIG. 1B, the conveyors may form one or more loops that convey a shuttle, a puck, and/or a pill container from one station to another in the APFS 100. Although shown with a main conveyor 104 operating in a counterclockwise loop (e.g., transferring material counterclockwise through the APFS 100), it should be appreciated that the stations may be arranged in such an order where the loop runs in an opposite direction (e.g., in a clockwise loop, etc.).

The APFS 100 may include one or more pill container sorting stations 108 where pill containers of various sizes are loaded into hoppers and arranged in a particular orientation onto a feed line, or lane. In some examples, the pill containers may be orientated by one or more vibratory feeder bowls, flexible feeders, or other sorting system. The pill container sorting station 108 may provide a lane or queue of pill containers starting from the sorting system and ending adjacent a labeling conveyor 103 (shown in FIG. 1B). In one example, each of the pill containers may be arranged such that an opening of the pill container is face up. The pill containers may also be referred to herein as a “vial,” “container,” “bottle,” and/or the like. The pill containers may include first-size pill containers having a first volume (e.g., 120 cubic centimeters) and second-size pill containers having a second volume (e.g., 200 cubic centimeters). These pill containers may be opaque, translucent (e.g., amber colored), and/or combinations thereof.

Once an order is initiated (e.g., when a pill order is received by the APFS 100), the appropriate size of pill container may be fed, placed, or otherwise moved onto the labeling conveyor 103 toward a labeling station 112. The size of the pill container may be determined based on prescription information, an amount of pills to be dispensed for the pill order, or some other information associated with the pill order.

Next, the pill container is moved into a labeling station 112. The labeling station 112 may comprise at least one label marking or label printing system. In one example, the label marking system may correspond to an inkjet, or similar, printer that is capable of spraying an ink label directly onto the outer surface of a pill container. Additionally or alternatively, the label printing system may include a printer that prints filling information or pill order information onto an adhesive label. In some examples, the label marking system may comprise a direct thermal printer and/or thermal printing system that is configured to prepare the label and/or other documentation that accompanies the pill order. The labeling station 112 may then apply the label directly to an outer surface of the pill container. In some examples, the pill container may be held in a special label application pallet that allows the pill container to contact a dispensed label and move past an application head that physically applies the label to the outer surface of the pill container. The label application pallet may allow the pill container to rotate as the pill container and label application pallet moves past the label application via the labeling conveyor 103. By way of example, the labeling station 112 may include four or more print-apply labelers utilizing a label printer. Continuing this example, the pill containers arrive at the labeling station 112 in a pallet or shuttle where they are labeled and rolled on as the shuttle travels past, for instance, a polyurethane pad.

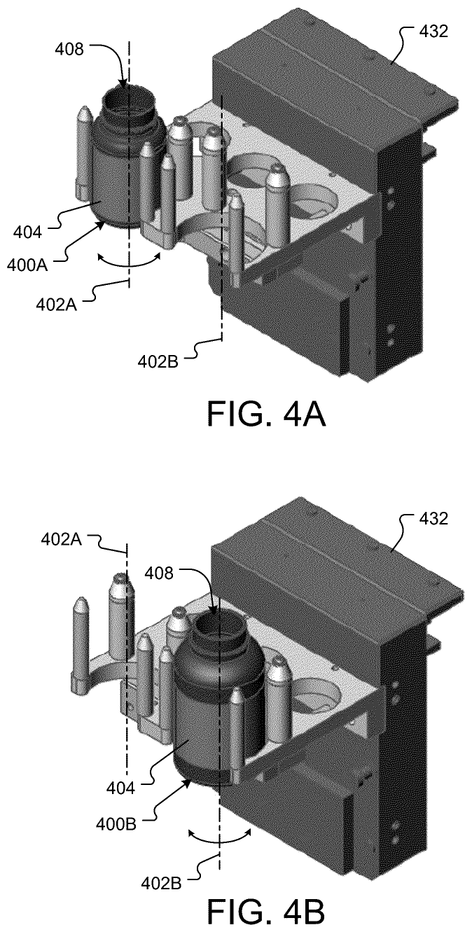

In one example, the label may correspond to an encoding of information that is printed onto the label (e.g., in the form of a barcode, text, etc., and/or combinations thereof). The information may be encoded digitally into an RFID tag associated with the label and/or the pill container. In some examples, the label application pallets may continuously move around a loop of the labeling conveyor 103. Additional details of one example of the label application pallets are described in conjunction with FIGS. 4A-4B.

Once a label is applied to the pill container, the APFS 100 may instruct a pick-and-place system, PnP, or other transfer mechanism, to move the pill container from the labeling conveyor 103 onto the main conveyor 104. The pick-and-place, PnP, may be arranged between or adjacent to the labeling conveyor 103 and the main conveyor 104. In any event, the pick-and-place, PnP, may move the pill container into a puck that is engaged with a shuttle of the main conveyor 104. The pick-and-place, PnP, may correspond to one or more linear actuators that are coupled together to grasp a pill container from a label application pallet, lift the pill container from the label application pallet, translate the pill container from a position at the labeling conveyor 103 into a position above a puck on the main conveyor 104, lower the pill container into the puck, and release the grasp of the pill container. Once the pill container is released into the puck, the pick-and-place, PnP, may return to the original position (e.g., ready to grasp another pill container for placement onto the main conveyor 104). The pick-and-place, PnP, may correspond to a robot or one or more pneumatic, hydraulic, and/or electromechanical actuators (e.g., air cylinders and slides, hydraulic cylinders and slides, or solenoids and slides) that are coupled together to move in two or more dimensions.

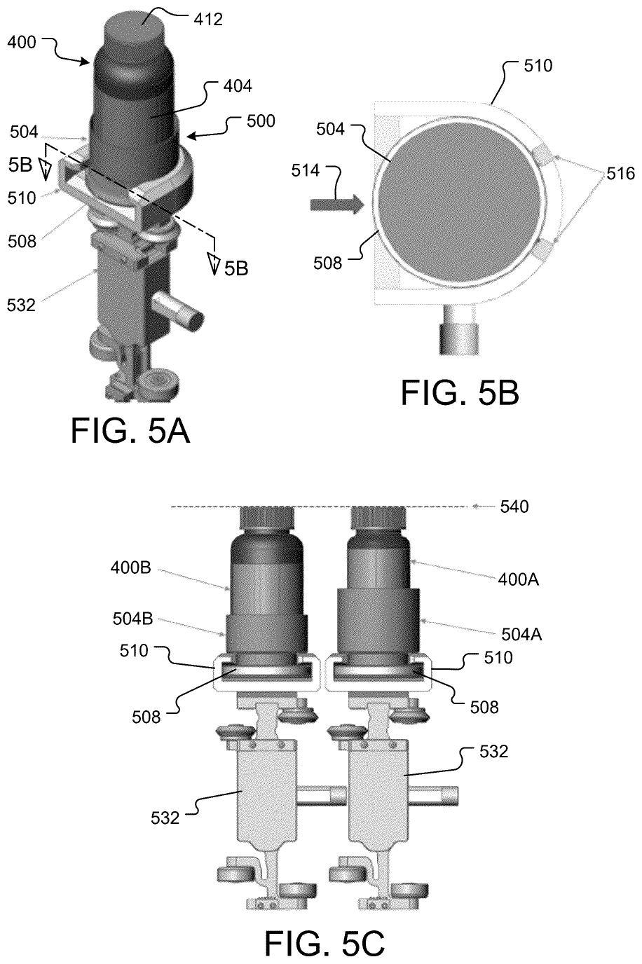

The pucks may be arranged by a puck accumulation station 116 to feed a number of pucks having a specific size into a position adjacent the main conveyor 104. The pucks may be fed out to a shuttle to prepare for the pill container in queue. In some examples, each of the shuttles may include a unique RFID tag. In one example, each puck may include a unique RFID tag. In any event, the pill containers may be placed into the pucks where they are “married” or otherwise coupled to the RFID tag. In one example, the puck accumulation station 116 may include a machine that arranges pucks of a first size in a first lane and that arranges pucks of a second size in a second lane. The end of each lane may be arranged adjacent the main conveyor 104. Based on the pill order received and the labeled pill container in queue on the labeling conveyor 103, the puck accumulation station 116 may engage a puck of a specific size onto a shuttle of the main conveyor 104. The shuttles of the main conveyor 104 may be configured to receive the pucks of the first size and the pucks of the second size utilizing a universal shuttle receiving area. The pucks may be selectively engaged and disengaged from the shuttles of the main conveyor 104 at one or more points along the length of the main conveyor 104. More details regarding the shuttles of the main conveyor 104 are described in conjunction with FIGS. 5A-5C. The shuttles are configured to move in a track along the main conveyor 104.

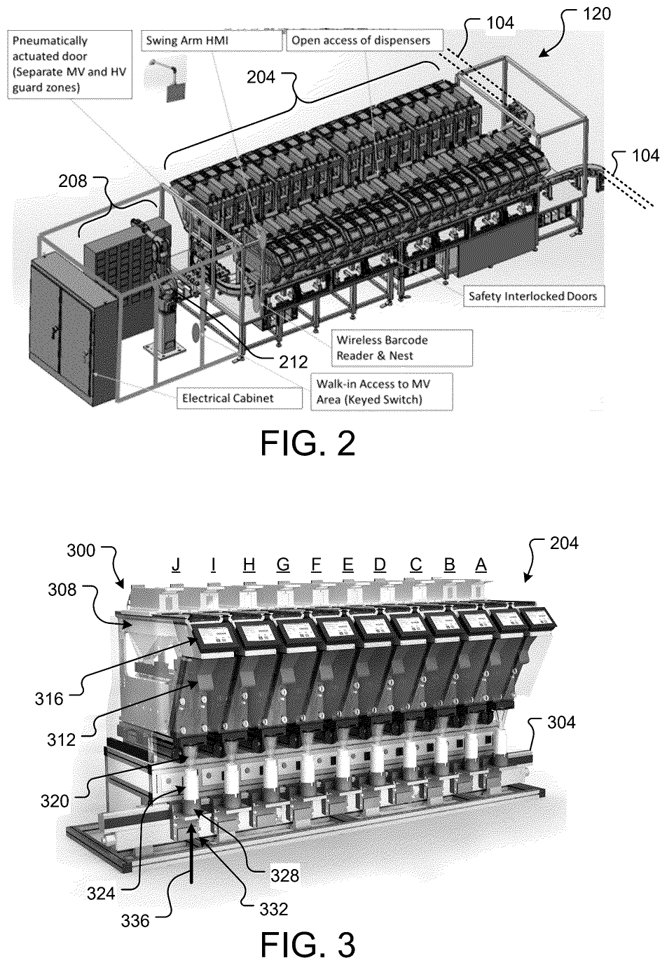

After a pill container is coupled with a corresponding puck and shuttle (e.g., based on the pill order), the puck and shuttle may convey the pill container to one or more dispensing and counting lanes 120A-120F. The dispensing and counting lanes 120A-120F may be disposed along a length of the main conveyor 104 side-by-side. The dispensing and counting lanes 120A-120F may include an array of high-volume automatic pill dispensers (e.g., a first array of automatic pill dispensers) and an array of low-volume automatic pill dispensers (e.g., a second array of automatic pill dispensers). Each of the automatic pill dispensers in the first or second array is configured to automatically dispense pills into a corresponding pill container based on information in the pill order. The automatic pill dispensers are described in further detail in conjunction with FIGS. 2-3. Among other things, the present disclosure describes a combination of inline high-volume automatic pill dispensers and offline low-volume automatic pill dispensers that are capable of simultaneously filling pill orders of all types, independently, without negatively affecting the speed of the APFS 100. Since each of the shuttles is allowed to move independently of one another along the track of the main conveyor 104 (and each of the spurs for pill dispensing, capping, consolidation, packaging etc.), and since the puck holding a pill container may be selectively engaged and disengaged with any shuttle, the APFS 100 can continue to move shuttles while pills are being dispensed in any one or more of the arrays of automatic pill dispensers.

In some examples, after the pill containers have been filled by one or more of the automatic pill dispensers in the dispensing and counting lanes 120A-120F, the pill containers may be imaged (e.g., prior to capping and/or sealing). Imaging may correspond to video and/or still images taken of the pill container, the contents of the pill container (e.g., pills dispensed therein), etc. The images may be selectively sent to a pharmacist to review and compare to an image of the pill associated with the pill order in the NDC directory. This comparison and/or review may be performed by a pharmacist, remotely located apart from the APFS 100, on a computer screen. Any anomalies or inconsistencies may be reported by the pharmacist (e.g., as a suspect pill container) and the pill container may be directed to a QA station 128 or reject area for further review and/or inspection. In some examples, a suspect pill container may be directed to the QA station 128 bypassing a capping station 124, etc. The imaging and pill review may be done using the designs and techniques described in U.S. patent application Ser. No. 17/330,803, entitled “System and Method for Imaging Pharmacy Workflow in a Virtual Verification System”, filed on May 26, 2021; U.S. patent application Ser. No. 17/330,813, entitled “System and Method for Virtual Verification in Pharmacy Workflow, filed on May 26, 2021; and U.S. patent application Ser. No. 29/785,558, entitled “Counting and Imaging Tray”, filed on May 26, 2021, each of which is incorporated by reference herein in its entirety.

The capping station 124 may apply a seal and/or cap to the open end of the pill container. The cap may seal the contents inside the pill container. The capping station 124 may utilize one or more automatic (e.g., electronic) torque systems and controllers. The capping station 124 may be configured to hold a cap (e.g., with a gripper, etc.), align the cap with the pill container, engage the cap with the pill container, and rotate the cap until the cap is screwed onto the pill container at a predetermined torque setting or value.

After capping, the pill containers have the option to enter the QA station 128 (e.g., quality assurance sampling and exception area, etc.) if needed. If not, the pill container will move onto consolidation, packing, and/or transfer to an existing pharmacy. The QA station 128 may be used to validate an accuracy of pill dispenses performed by one or more of the automatic pill dispensers in the first and/or second arrays of automatic pill dispensers. The QA station 128 may be configured to randomly verify fills, or routinely verify fills, to qualify a particular machine, automatic pill dispenser, or array of automatic pill dispensers.

The APFS 100 may utilize a consolidation station 132 that dynamically manages shuttles to group pill orders and/or prescriptions. To minimize sort times, multiple prescriptions that are associated with a particular pill order (e.g., pill containers containing respective prescriptions that form a part of a greater pill order for a customer, etc.) may be placed in adjacent shuttles and travel directly to the packaging station 140 as a complete order. Any orders that do not need to be consolidated (e.g., single prescription or pill container associated with a pill order, etc.) may be routed directly to the packaging station 140, for example, without entering the consolidation station 132. On the other hand, orders that have not yet been consolidated on the fly will enter the consolidation area to await their mates (e.g., other pill containers forming a complete pill order, etc.).

The APFS 100 may utilize a bagging station 136 that is arranged after the capping station 124. The bagging station 136 may correspond to a semi-automatic packaging station. The bagging station 136 may be configured for pill orders having greater than a predetermined number of pill containers (e.g., eight or more, etc.), and/or for pill orders that exceed a predetermined number of pages (e.g., ten pages, etc.) of paperwork.

In some examples, the APFS 100 may include a “unload to legacy” station, shown disposed between the bagging station 136 and the packaging station 140. Since all pill orders may not be pill container only, those with marriages (e.g., having one or more accompanying products) from other zones, lines, or fulfillment areas may be offloaded from the APFS 100 and travel to those fulfillment areas, via one or more conveyors, for further processing and/or fulfillment.

The packaging station 140 may correspond to a multiple pill container packaging system. The APFS 100 may utilize separate spurs for automatically packing one or more pill containers (e.g., 1 pill container to 8 pill container, etc.). Each spur may include a plurality of baggers and a plurality of printers. These baggers and printers may each be capable of 3,000 orders per hour, or more. The packaging station 140 may, based on information in the pill order, print one or more inserts (or sheets) that are inserted into a bag along with the filled and capped pill container associated with the pill order. More specifically, the pill container may be removed from a respective puck via a pick-and-place, PnP, and the empty puck (e.g., puck without pill container) and shuttle may return to the puck accumulation station 116. At the puck accumulation station 116, the puck may be removed from the shuttle and the shuttle may index to a position ready to receive another different puck. In some examples, the empty puck may join a queue of pucks in the puck accumulation station 116 awaiting replacement onto a shuttle for another order. The empty pucks may be arranged and/or accumulated based on size (e.g., the size of the pill container that the puck is configured to receive). In any event, the fulfilled and bagged pill order may be sent from the packaging station 140 to a package sortation and shipping process area, staging area, and/or to a carrier for delivery to a recipient address associated with the pill order. In some examples, the packaging station 140 may include an automated “variable bag sizing” capability. For example, a smaller bag may be utilized for one-bottle and two-bottle (e.g., one-container and two-container, etc.) orders and a larger bag may be utilized for orders containing more than two bottles/containers. In one example, this variable bag sizing may include “plug & play” bagging automation technology.

FIG. 1B shows a second schematic plan view of the APFS 100 in accordance with examples of the present disclosure. In particular, the APFS 100 is shown in FIG. 1B as including a labeling conveyor 103 arranged at a first end 102A of the main conveyor 104 and/or APFS 100. The labeling conveyor 103 is shown, by solid arrows, as indexing pill containers in a counterclockwise loop from the pill container sorting station 108 to the pick-and-place, PnP. As described above, the pick-and-place, PnP, is arranged adjacent the labeling conveyor 103 and the main conveyor 104 at the first end 102A. The main conveyor 104 is shown having a track that moves from the first end 102A to the second end 102B, along the first side 106A, and then back to the first end 102A from the second end 102B, along the second side 106B, in a main conveyor loop, shown in dashed line arrows. As illustrated in FIG. 1B, the main conveyor loop runs counterclockwise providing at least one travel path for a pill container to follow from the first end 102A through the dispensing and counting lanes 120A-120F, through the capping station 124, the QA station 128, the consolidation station 132, the bagging station 136, and/or the packaging station 140. In some examples, the conveying of shuttles with the pill containers inside the pucks travel into and out of several conveyor spurs that may be identical to the main loop. The spurs may include pill dispensing, capping, packaging etc. The travel path may be configured as an uninterrupted, or continuous, loop. In some examples, a shuttle, whether including or not including a puck and/or pill container, may bypass one or more sections or stations of the APFS 100.

One or more identification readers (e.g., barcode readers, RFID readers, etc.) may be arranged along the length of the track of the main conveyor 104 and around various points (e.g., conveyor spurs) of the loop of the main conveyor 104. These identification readers are shown as circles comprising a shaded interior fill. As illustrated in FIG. 1B, an identification reader may be disposed before, or adjacent, each station (e.g., shown as solid dark filled circles). The main conveyor 104 may include a built-in encoder that provides precision placement of each shuttle through the entire conveying loop and the spurs. In some examples, the main conveyor 104 may have the ability to accelerate and decelerate the shuttle in one or more sections (e.g., along the various lengths of the main conveyor 104). The encoding may be continuous throughout the entire conveying system and may not be limited to the points (e.g., the circles) as illustrated in FIG. 1B. In one example, the APFS 100 may not read the barcode on the pill container label at these points. Stated another way, instructions from the control system in conjunction with the RFID tag in the shuttle may provide the instruction of where the shuttle travels for each path in the entire system. Alternatively, as a pill container moves into a detection, or reading, proximity of a particular identification reader, the identification reader may read the label of the pill container and the APFS 100 may determine to direct the pill container to one or more stations, dispensing and counting lanes 120A-120F, etc., in the APFS 100. In some examples, one or more of the dispensing and counting lanes 120A-120F may include an identification reader that, upon reading a label of a pill container, may cause the pill container to be moved (e.g., separate or apart) from the main conveyor 104 to a low-volume (e.g., offline) automatic pill dispenser array. The APFS 100 may move the pill container via one or more robots, actuators, conveying systems, encoder devices (e.g., to route the shuttles, etc.), and/or the like.

FIG. 1C shows a block diagram of the APFS 100 in accordance with examples of the present disclosure. The APFS 100 may include one or more of a main controller 154, a conveyor controller, a memory 158, a user interface 162, a network interface 166, conveyor actuator(s) 170, fulfillment stations 174, identification or barcode reader(s) 194, and/or the like, that are interconnected to one another via one or more bus 152 (e.g., power, communications, and/or combinations thereof). Each of the fulfillment stations 174 may comprise a respective processor 178, station hardware 182, station memory 186, station instructions 190, etc. Each of the fulfillment stations 174 may communicate with the main controller 154 or vice versa. Additionally or alternatively, each of the fulfillment stations 174 may communicate with one another in the APFS 100.

The bus 152 may correspond to a power and/or a communications bus. In accordance with at least some examples of the present disclosure, the bus 152 may comprise any type of known communication medium or collection of communication media and may use any type of protocols to transport messages between endpoints. The bus 152 may include wired and/or wireless communication technologies. The Internet is an example of the bus 152 that constitutes an Internet Protocol (“IP”) network consisting of many computers, computing networks, and other communication devices located all over the world, which are connected through many telephone systems and other means. Other examples of the bus 152 include, without limitation, a standard Plain Old Telephone System (“POTS”), an Integrated Services Digital Network (“ISDN”), the Public Switched Telephone Network (“PSTN”), a Local Area Network (“LAN”), a Wide Area Network (“WAN”), a VoIP network, a cellular network, and any other type of packet-switched or circuit-switched network known in the art. In addition, it can be appreciated that the bus 152 need not be limited to any one network type, and instead may be comprised of a number of different networks and/or network types. The bus 152 may comprise a number of different communication media such as coaxial cable, copper cable/wire, fiber-optic cable, antennas for transmitting/receiving wireless messages, optical/infrared, encoder technology, and combinations thereof.

The main controller 154 may correspond to a controller or processor that is capable of executing instructions causing the APFS 100 to fulfill pill orders received from an ordering entity. The main controller 154 may send instructions to one or more of the components and/or fulfillment stations 174 of the APFS 100. In some examples, the main controller 154 may correspond to one or more computer processing devices. For example, the main controller 154 may be provided as silicon, an Application-Specific Integrated Circuit (“ASIC”), as a Field Programmable Gate Array (“FPGA”), any other type of Integrated Circuit (“IC”) chip, a collection of IC chips, and/or the like. In some examples, the main controller 154 may be provided as a Central Processing Unit (“CPU”), a microprocessor, or a plurality of microprocessors that are configured to execute the instructions sets stored in memory 158. Upon executing the instruction sets stored in memory 158, the main controller 154 enables various communications, convey signals, dispense signals, and/or interaction functions of the APFS 100 and/or fulfillment stations 174, etc., and may provide an ability to establish and maintain communication sessions between communication devices over the bus 152 when specific predefined conditions are met. The main controller 154 may be embodied as a virtual processor(s) executing on one or more physical processors. The execution of a virtual processor may be distributed over a number of physical processors or one physical processor may execute one or more virtual processors. Virtual processors are presented to a process as a physical processor for the execution of the process while the specific underlying physical processor(s) may be dynamically allocated before or during the execution of the virtual processor wherein the instruction stack and pointer, register contents, and/or other values maintained by the virtual processor for the execution of the process are transferred to another physical processor(s). As a benefit, the physical processors may be added, removed, or reallocated without affecting the virtual processors execution of the processes. For example, main controller 154 may be one of a number of virtual processors executing on a number of physical processors (e.g., “cloud,” “farm,” “array,” etc.) and presented to the processes herein as a dedicated processor. Additionally or alternatively, the physical processor(s) may execute a virtual processor to provide an alternative instruction set as compared to the instruction set of the virtual processor (e.g., an “emulator”). As a benefit, a process compiled to run a processor having a first instruction set (e.g., Virtual Address Extension (“VAX”)) may be executed by a processor executing a second instruction set (e.g., Intel® 9xx chipset code, etc.) by executing a virtual processor having the first instruction set (e.g., VAX emulator).

The memory 158, or storage memory, may correspond to any type of non-transitory computer-readable medium. In some examples, the memory 158 may comprise volatile or non-volatile memory and a controller for the same. Non-limiting examples of the storage memory 158 that may be utilized in the main controller 154 and/or APFS 100 may include Random Access Memory (“RAM”), Read Only Memory (“ROM”), buffer memory, flash memory, solid-state memory, or variants thereof. Any of these memory types may be considered non-transitory computer memory devices even though the data stored thereby can be changed one or more times. The memory 158 may be used to store information about pill orders, communications, identifications, conditional requirements, times, authentication, authorization, validation, compliance, history, and/or the like. In some examples, the memory 158 may be configured to store rules and/or the instruction sets depicted in addition to temporarily storing data for the main controller 154 to execute various types of routines or functions. Although not depicted, the memory 158 may include instructions that enable the main controller 154 to store data into a memory storage device and retrieve information from the memory storage device. In some examples, the memory storage device or the data stored therein may be stored internal to the APFS 100 or main controller 154 (e.g., within the memory 158 of the main controller 154 and/or the APFS 100 rather than in a separate database) or in a separate server.

The user interface 162 may correspond to any type of input and/or output device, or combination thereof, that enables a user to interact with the APFS 100. As can be appreciated, the nature of the user interface 162 may depend upon the nature of the components of the APFS 100. Examples of the user interface 162 may include, but are in no way limited to, user interface hardware and devices such as at least one touch-sensitive display elements, buttons, switches, keyboards, peripheral interface devices (e.g., mice, controller, joysticks, etc.), human-machine interface (“HMI”), and/or the like. It is an aspect of the present disclosure that one or more devices in the user interface 162 may provide an input that is interpreted by the main controller 154 in controlling one or more components of the APFS 100.

The network interface 166 provides the APFS 100 with the ability to send and receive communication packets or the like over the bus 152 and/or over some other communication network. The network interface 166 may be provided as a network interface card (“NIC”), a network port, a modem, drivers for the same, and the like. Communications between the components of the APFS 100 and other devices connected to the bus 152 may flow through the network interface 166 of the APFS 100. Examples of a suitable network interface 166 may include, without limitation, an antenna, a driver circuit, an Ethernet port, a modulator/demodulator, an NIC, an RJ-11 port, an RJ-45 port, an RS-232 port, a USB port, etc. The network interface 166 may include one or multiple different network interfaces depending upon whether the APFS 100 is connecting to a single communication network or multiple different types of communication networks. For instance, the APFS 100 may be provided with both a wired network interface and a wireless network interface without departing from the scope of the present disclosure. In some examples, the APFS 100 may include different communications ports that interconnect with various input/output lines.

The conveyor actuator(s) 170 may correspond to any actuator that powers a shuttle along the track of the main conveyor 104 via an electrical or electromagnetic signal. In one example, the conveyor actuator(s) 170 may include encoder technology and may correspond to a magnetically energized shuttle and conveyor system. In this example, a convey signal sent by the main controller 154 to the conveyor actuator(s) 170 may cause one or more shuttles to move along the track of the main conveyor 104 to one or points disposed along the length of the main conveyor 104. The conveyor actuator(s) 170 may be configured to send one or more convey signals to move one shuttle in the track of the main conveyor 104 independently of other shuttles in the track of the main conveyor 104.

Each of the fulfillment stations 174 of the APFS 100 may include at least one of a respective processor 178, station hardware 182, station memory 186, station instructions 190, and/or the like. The fulfillment stations 174 may correspond to any one or more of the stations described in conjunction with FIGS. 1A-1B, and elsewhere herein. For example, the fulfillment stations 174 may correspond to components in the pill container sorting station 108, the labeling station 112, the puck accumulation station 116, a dispensing and counting lane 120A-120F, the capping station 124, the QA station 128, the consolidation station 132, the bagging station 136, the packaging station 140, and the like.

The processor 178 may be similar, or identical, to the main controller 154. In some examples, the processor 178 and/or the main controller 154 may correspond to a programmable logic controller (“PLC”) and/or a synchronous logic controller (“SLC”), etc. In one example, the processor 178 may receive instructions, signals, and/or the like from the main controller 154 to perform an operation of a fulfillment station 174 that is controlled by the processor 178 of the respective fulfillment station 174.

The station hardware 182 may correspond to electrical, mechanical, and/or electromechanical subsystems associated with each of the fulfillment stations 174. This station hardware 182 may include actuators, encoder technology, solenoids, valves, sensors, cameras, labelers, printers, and/or more.

The station memory 186 may be similar, or identical, to the memory 158. The station memory 186 may be configured to store rules and/or the instruction sets depicted in addition to temporarily storing data for processor 178 to execute various types of routines or functions for the respective fulfillment station 174. The station instructions 190 may correspond to operation instructions and/or information associated with each fulfillment station 174 that, among other things, allow each of the fulfillment stations 174 to operate independently of one another.

The identification or barcode reader(s) 194 may correspond to any number of identification reader that is capable of automatically reading information associated with a pill container (e.g., embedded on a label, RFID tag, optical tag, etc.) that is in proximity to the barcode reader(s) 194. In some examples, the barcode reader(s) 194 may be configured as RFID readers that can read information associated with the pill container, the puck, the shuttle, and/or combinations thereof. The RFID tag in the shuttle may include a unique license identification of the shuttle. The conveyor control system may assign the shuttle to deliver the respective pill container and puck within the shuttle, to the designated pill dispense location or other location in the system. In some examples, any of the tags and readers described herein may include optical tags and optical readers. Examples of optical tags may include, but are in no way limited to, infrared inks, magnetic inks, ultraviolet fluorescent compounds, and/or the like. The optical tags may be passive, reactive, or active. The optical tagging and reading technology that may be used in place of, or in conjunction with, the tags and readers described herein may correspond to one or more devices methods and systems as described in the July 2003 Sandia Report entitled, “A Brief Examination of Optical Tagging Technologies” by Mark R. Ackermann, Paul A. Cahill, Timothy J. Drummond and Jess P. Wilcox, published by Sandia National Laboratories, the entirety of which is hereby incorporated herein by reference, in its entirety, for all that it teaches and for all purposes.

FIG. 2 shows a perspective view of multiple arrays of automatic pill dispensers 204, 208 in accordance with examples of the present disclosure. As shown, the arrays of automatic pill dispensers 204, 208 may form part of a dispensing and counting lane 120 in the APFS 100. The dispensing and counting lane 120 may correspond to any one or more of the dispensing and counting lanes 120A-120F described in conjunction with FIGS. 1A-1B. The dispensing and counting lane 120 may be part of, or attached to, the main conveyor 104 of the APFS 100. Stated another way, the shuttles moving along the main conveyor 104 may be directed to a particular dispensing and counting lane 120 disposed along the main conveyor 104. In some examples, two low volume dispenser units may be arranged at the end of each dispensing and counting lane 120.

The dispensing and counting lane 120 is shown as being separated into a high-volume zone and a low-volume zone. The high-volume zone includes a first array of automatic pill dispensers 204 that are configured to dispense popular (e.g., most commonly dispensed, etc.) pills in the APFS 100. The low-volume zone includes a second array of pill dispensers 208 that are configured to dispense less popular (e.g., less commonly dispensed, etc.) pills than the first array of automatic pill dispensers 204 in the APFS 100. The second array of pill dispensers 208 may be arranged “offline” or separate and apart from the main conveyor 104. In one example, a location may be arranged directly adjacent the conveyor spur where the shuttle will deliver a puck. In some examples, a robot 212 (e.g., a six-axis robot, gantry robot, pick-and-place, etc.) may be arranged adjacent a portion of the main conveyor 104 and/or end loop of the dispensing and counting lane 120. The robot 212 may be configured to transfer a pill container and/or a puck from a shuttle on the main conveyor 104 into a second array of pill dispensers 208. In one example, the pill container may be lifted out of the puck to the specific pill dispense location by the robot 212. Upon completion of the dispense, the robot 212 may move the pill container back into the puck. Immediately after this step, the puck may be joined to a shuttle where the conveyance system will bring that shuttle to the next step in the process. In one example, the second array of pill dispensers 208 may dispense pills at a rate of speed that is lower than the first array of automatic pill dispensers 204. Accordingly, the second array of pill dispensers 208 may operate independently of the first array of automatic pill dispensers 204. Moreover, the first array of automatic pill dispensers 204 may continue to operate and dispense pills while the second array of pill dispensers 208 is dispensing pills offline, allowing shuttles to move along the main conveyor 104 unimpeded by any shuttle waiting for the offline dispensing to be performed. In one example, the shuttle may not be waiting for the dispense. In this example, the shuttle may depart from the puck once it is delivered to the area adjacent the second array of pill dispensers 208. Once the dispense is complete and the robot 212 places the filled pill container into the puck, another shuttle may pick up the filled pill container and puck. For instance, the APFS 100 may send a shuttle and/or puck to a point adjacent the robot 212 when the second array of pill dispensers 208 has performed a fill operation and is ready to transfer the filled pill container to the main conveyor 104. The dispensing and counting lane 120 may include one or more electrical cabinets. The electrical cabinet may house one or more components of the fulfillment stations 174 and/or the APFS 100 (e.g., the main controller 154, the bus 152, etc.). Among other things, the arrangement of each dispensing and counting lane 120 in the APFS 100 may allow for refilling of the first array of automatic pill dispensers 204 and/or the second array of pill dispensers 208, while the APFS 100 is running (e.g., without machine interruption, etc.).

FIG. 3 shows a perspective view of the first array of automatic pill dispensers 204 in accordance with examples of the present disclosure. Each automatic pill dispenser 300 in the first array of automatic pill dispensers 204 may be arranged adjacent to another along a lane conveyor section 304 of the main conveyor 104. The automatic pill dispenser 300 may include a pill magazine 308, an image sensor 312, a user interface 316, and a dispense funnel 320. The pill magazine may contain a plurality of pills to be dispensed by the automatic pill dispenser 300. The image sensor 312 may correspond to a camera that is configured to at least detect and recognize a color and a shape of pills dispensed. In some examples, the camera may recognize text or decorations applied to a portion of each pill being dispensed. The image sensor (e.g., vision camera, etc.) may be located just above the dispense funnel 320 at an area before the pills move downward through the dispense funnel 320. A shuttle 332 and pill container 324 may be arranged adjacent the dispense funnel 320 prior to filling. Once aligned, the material handling puck 328 holding the pill container 324 may raise in a lift direction 336 toward the dispense funnel 320 such that a portion of the dispense funnel 320 is arranged inside the opening of the pill container 324. Stated another way, the dispense funnel 320 and the pill container 324 are placed together with no gap between the two openings. The pill container 324 may be raised to the dispense funnel 320, and the dispense funnel 320 may be stationary. Additionally or alternatively, the dispense funnel 320 may move in a direction toward the pill container 324 such that the portion of the dispense funnel 320 is engaged with the opening of the pill container 324. In any event, the arrangement/engagement of the dispense funnel 320 with the opening of the pill container 324 ensures that no pills escape, or are spilled, during a dispense operation. After the pill container 324 is filled, the dispense funnel 320 and the pill container 324 may be disengaged from one another (e.g., providing a space between the dispense funnel 320 and the pill container 324), allowing the shuttle 332 to move to the next station in the APFS 100. In some examples, the shuttle may deliver the empty pill container 324 with the material handling puck 328 to a designated pill dispenser 300, then departs to the next assigned task. In this example, another (e.g., different) shuttle 332 may pick up the filled pill container 324 and material handling puck 328 after the dispense is complete and will deliver the filled pill container 324 to the next assigned task.

FIGS. 4A-4B show perspective views of a pill container labeling shuttle 432 of a labeling conveyor holding a first size of pill container 400A and second size of pill container 400B, respectively, in accordance with examples of the present disclosure. The labeling shuttle 432 may include two or more nest locations that are configured to receive pill containers 400A, 400B of various sizes. Each pill container 400A, 400B includes a pill container opening 408 and an outer surface to which a label 404 may be applied. The nest of the labeling shuttle 432 allows the first size of pill container 400A to rotate about a first rotational axis 402A while the label 404 is applied to the outer surface thereof. The nest of the labeling shuttle 432 allows the second size of pill container 400B to rotate about a second rotational axis 402B while the label 404 is applied to the outer surface thereof. In some examples, each labeling shuttle 432 may contain only one of the two pill container sizes 400A, 400B at a time, but not both at the same time. Stated another way, the labeling shuttles 432 may be universal and configured to either hold one small bottle (e.g., first size of pill container 400A) or one large bottle (e.g., second size of pill container 400B) at a time, but not both at the same time. The nest of the labeling shuttle 432 may include a plate with pins extending therefrom, the pins contacting an outer periphery of the body of each pill container 400A, 400B. The pins may allow a portion of the outer periphery of the body of each pill container 400A, 400B to protrude a further distance from the first and second rotational axes 402A, 402B than a distance between the first and second rotational axes 402A, 402B and the pins.

FIG. 5A shows a perspective view of main conveyor shuttle 532 holding a pill container in a puck 500 according to examples of the present disclosure. The shuttle 532 may correspond to an electrically or electromagnetically actuated carriage including one or more bearings and/or wheels and a nest 510 arranged thereon. The nest 510 may be configured to receive a puck 500 that includes a puck body 504 and a base ring 508. The nest 510 may include a lead-in, or chamfered, taper along a first edge that allows quick and error-free insertion into a receiving area thereof. As illustrated in FIG. 5A, the pill container 400 comprising a label 404 and a cap 412 is shown engaged with the puck 500. In this example, the puck 500 is shown engaged with the nest 510 of the shuttle 532.

FIG. 5B shows a section top plan view taken along line 5B-5B of the pill container and puck 500 shown in FIG. 5A. The puck 500 may be selectively engaged with and disengaged with the nest 510 of the shuttle 532. To engage the puck 500 with the nest 510, the puck 500 is moved, or slid, in the puck insertion direction 514 into the receiving area of the nest 510. Once the base ring 508 is moved into a fully engaged position, the magnet(s) 516 in the nest 510 may magnetically attract the pill container sorting station 108. In some examples, the base ring 508 may be made from, or include, a ferritic or magnetic material. The base ring 508 may be made from a ferritic stainless steel such as, but in no way limited to, 420 stainless steel, 440 stainless steel, 17-4 phenolic stainless steel, and/or the like. The magnet(s) 516 may be configured to hold the puck 500 in place during movement of the shuttle 532 throughout the APFS 100. When the puck 500 is disengaged from the shuttle 532 (e.g., the nest 510 of the shuttle 532), the puck 500 is moved in a direction opposite the puck insertion direction 514.

FIG. 5C shows a side elevation view comparing a fill height for a pill container shuttle 532 with a first puck body size 504A and first pill container size 400A and a second puck body size 504B and second pill container size 400B in accordance with examples of the present disclosure. The pucks 500 may be arranged in one or more sizes. Although each of the pucks 500 may include a common, or universal, base ring 508 and nest engagement features, the puck body 504 may differ based on a corresponding size of the pill container 400. For instance, the first puck body size 504A may be configured to receive the first pill container size 400A (e.g., 120 cubic centimeter volume) and the second puck body size 504B may be configured to receive the second pill container size 400B (e.g., 200 cubic centimeter volume). The first puck body size 504A and the second puck body size 504B may be arranged such that, regardless of the size of the pill container 400, the top of the pill container 400 is arranged along a common fill height line 540, or fill height plane. As can be appreciated, this arrangement allows a single shuttle 532 to handle pill containers 400 of any size and resulting in a simplified and efficient automated arrangement.

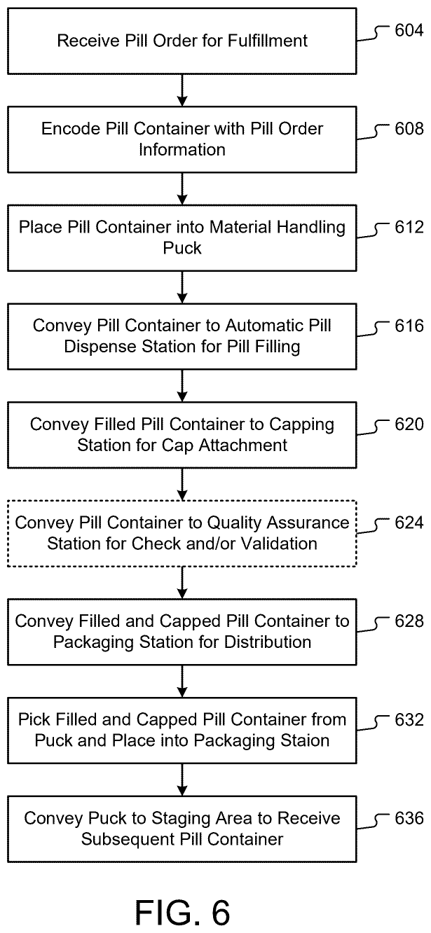

FIG. 6 is a flow diagram of a method for automatically filling containers using the APFS 100 in accordance with examples of the present disclosure. The method can be executed as a set of computer-executable instructions (e.g., the system and/or station instructions 190, etc., previously described, etc.) executed by a computer system (e.g., the APFS 100, the main controller 154, the processor 178, etc.) and encoded or stored on a computer readable medium (e.g., the memory 158, the station memory 186, etc.). Hereinafter, the method shall be further explained with reference to the systems, components, modules, applications, software, user interfaces, etc. described in conjunction with FIGS. 1A-5C.

The method may begin by receiving pill order from one or more ordering endpoints (step 604). The pill order may include information such as a particular pill type, number of pills, prescription information, and/or patient information. The pill order may be received from a doctor, pharmacy, care center, hospital, end user, patient, and/or the like. The pill order may be received by the main controller 154 and stored in the memory 158.

Next, the method may proceed by encoding a specific pill container 400 with the pill order information (step 608). The specific pill container 400 may be selected based on information in the pill order. The pill container 400 may be selected based on a number of pills to be dispensed, a size of the pills to be dispensed, and/or the like. The pill container 400 may be selected based on interior volume. Encoding the information onto the pill container 400 may include, but is in no way limited to, printing a barcode on a label that is applied to the pill container 400 (e.g., by the labeling station 112), digitally encoding the information onto an RFID tag, and/or the like. In some examples, the pill container 400 may not be encoded with the pill order information. Each subsystem (e.g., label system, dispense system, packaging system, etc.) may receive information for the pill bottle task that needs to be performed when the specific pill container 400 enters the specific subsystem) and when pill order information is received from the control system.

Once the pill container 400 has been labeled with a unique barcode, or otherwise encoded, the pill container 400 is transferred from the labeling conveyor 103 onto a corresponding puck 500 (step 612). In some examples, the puck 500 may be encoded with the pill order information by writing data to an RFID associated with the puck 500. In any event, the pill order information may be used to route the pill container 400, puck 500, and/or the shuttle 532 throughout the APFS 100. Alternatively, the individual subsystems may receive instructions for a given task to be performed with a specific pill container 400. In this case, the order information may not be contained in the barcode, puck 500, or the shuttle 532 that transfers the puck 500.

Next, based on the pill order information, the pill container 400 is conveyed via the main conveyor 104 to the first array of automatic pill dispensers 204 or the second array of pill dispensers 208. When the pill container 400 is conveyed to the first array of automatic pill dispensers 204, the pill container 400 is positioned adjacent a dispense funnel 320 and then engaged therewith to ensure error-free filling. In some cases, the pill container 400 may be required to be conveyed to a second array of pill dispensers 208 instead of the first array of automatic pill dispensers 204. In this example, the pill container 400 may be conveyed by the shuttle 532 to an area adjacent a robot 212 and the second array of pill dispensers 208. The robot 212 may remove the pill container 400 and/or the puck 500 holding the pill container 400 from the shuttle 532 and place the pill container 400 and/or the puck 500 into one of a second array of pill dispensers 208 to be processed. As the second array of pill dispensers 208 fulfills the pill order (e.g., dispenses pills), the shuttle 532 may be allowed to move along the track of the main conveyor 104 back to the puck accumulation station 116 (e.g., to receive another puck or await further conveying instructions). For high volume NDC's multiple dispensers may be provided to accommodate the volume of pill containers 400 that need to be filled.

Once filled, the pill container 400 may pass through an imaging station that is configured to take images such as pictures and/or videos of the contents of the pill container 400. As provided above, this image information may be used by a pharmacist to ensure accuracy of the pill order, the fill operation, and/or automatic pill dispenser performance. The image information may be sent by the APFS 100 across a communication network to the pharmacist who can compare the pill order information and images to information stored in the NDC directory. Any anomalies may be marked or identified as suspect and the suspect pill container may be conveyed to a QA station 128 for further review.

The filled pill container 400 may be conveyed, via the main conveyor 104, to a capping station 124 for capping (step 620). The pill container 400 may be conveyed to the capping station 124 where a cap 412 is applied to the opening of the pill container 400, sealing the pills therein.

As provided above, the filled pill container 400 may be optionally conveyed to the QA station 128 for quality assurance review and/or exception (step 624). In some examples, this step may be utilized when first setting up an array of automatic pill dispensers and verifying or validating their operation.

The filled and capped pill containers 400 may be conveyed, via the main conveyor 104, to the packaging station 140 for distribution (step 628). The packaging station 140 may print one or more inserts to be included with the pill order. These inserts may include instructions for use, dosage information, patient information (e.g., identification, address, etc.), warnings, side effects, and/or the like. The packaging station 140 may remove the filled and capped pill container 400 from the puck 500 and shuttle 532 and then place the filled and capped pill container 400 into a package with the insert(s) (step 632). The packaging station 140 may include one or more barcode readers, for example, to verify each pill container 400 and printed document set immediately prior to the insertion into the bag or shipping container. Once packaged, the filled pill order may be sent to a recipient associated, or identified, with the pill order.

The puck may then be conveyed to a staging area and/or puck accumulation station 116 to be recirculated and/or reassigned to another pill order in the APFS 100 (step 636). More specifically, the shuttle 532 holding the empty puck 500 may be directed to the puck accumulation station 116 where a pick-and-place, or other actuator, removes the empty puck 500 from the shuttle 532. The puck 500 may then be placed into a queue based on size, need, and/or identification.

Any of the steps, functions, and operations discussed herein can be performed continuously and automatically.

While the flowcharts have been discussed and illustrated in relation to a particular sequence of events, it should be appreciated that changes, additions, and omissions to this sequence can occur without materially affecting the operation of the disclosed examples, configuration, and aspects.

The exemplary systems and methods of this disclosure have been described in relation to automated fulfillment systems and methods. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

References in the specification to “one example,” “an example,” “some examples,” etc., indicate that the example described may include a particular feature, structure, or characteristic, but every example may not necessarily include the particular feature, structure, or characteristic. Moreover, such phrases are not necessarily referring to the same example. Further, when a particular feature, structure, or characteristic is described in conjunction with one example, it is submitted that the description of such feature, structure, or characteristic may apply to any other example unless so stated and/or except as will be readily apparent to one skilled in the art from the description. The present disclosure, in various examples, configurations, and aspects, includes components, methods, processes, systems and/or apparatus substantially as depicted and described herein, including various examples, subcombinations, and subsets thereof. Those of skill in the art will understand how to make and use the systems and methods disclosed herein after understanding the present disclosure. The present disclosure, in various examples, configurations, and aspects, includes providing devices and processes in the absence of items not depicted and/or described herein or in various examples, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more examples, configurations, or aspects for the purpose of streamlining the disclosure. The features of the examples, configurations, or aspects of the disclosure may be combined in alternate examples, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed example, configuration, or aspect. Thus, the following claims are hereby incorporated into this Detailed Description, with each claim standing on its own as a separate preferred example of the disclosure.

Moreover, though the description of the disclosure has included description of one or more examples, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative examples, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Exemplary aspects are directed to an automated pill order filling system, including a main conveyor comprising a first end and a second end, wherein the main conveyor comprises a track running from the first end to the second end; a plurality of pucks configured to travel on the track (e.g., within a shuttle), wherein each puck of the plurality of pucks comprises a receiving area configured to hold a respective pill container; a first array of automatic pill dispensers disposed along a length of the track between the first end and the second end, wherein each automatic pill dispenser in the first array of automatic pill dispensers is configured to dispense a first number of pills into a first designated pill container at a first fill rate; a second array of automatic pill dispensers disposed adjacent the first array of automatic pill dispensers, wherein each automatic pill dispenser in the second array of automatic pill dispensers is configured to dispense a second number of pills into a second designated pill container at a second fill rate and is further configured to operate independently of the first array of automatic pill dispensers, and wherein the second fill rate is lower than the first fill rate; a processor; and a memory coupled with and readable by the processor and storing therein instructions that, when executed by the processor, cause the processor to: receive, via a communication network, a pill order comprising a specific number of pills that are associated with a specific pill type; encode at least one of a specific puck and a specific pill container with filling information for the pill order, wherein the filling information includes routing instructions for the specific pill container in the automated pill order filling system; send a first convey signal to the main conveyor, based on the routing instructions, to convey the specific puck and the specific pill container to a select automated pill dispenser selected from the first array of automatic pill dispensers or the second array of automatic pill dispensers; and send a dispense signal to the select automated pill dispenser to dispense the specific number of pills into the specific pill container in the specific puck forming a filled pill container.

Any one or more of the above aspects include wherein a capping station disposed along the length of the track between the first end and the second end, wherein the capping station is configured to attach a cap to a pill container filled with pills, and wherein the instructions that, when executed by the processor, further cause the processor to: send a second convey signal to the main conveyor to convey the filled pill container to the capping station; and send an attach signal to the capping station to attach a specific cap to the filled pill container. Any one or more of the above aspects include wherein each automatic pill dispenser in the first array of automatic pill dispensers comprises: a canister that is configured to receive and hold a plurality of pills; an actuator configured to move the plurality of pills from the canister through a dispense imaging area to a dispense funnel; and an image sensor arranged adjacent to the dispense imaging area, the image sensor configured to count each pill of the plurality of pills moving through the dispense imaging area. Any one or more of the above aspects include wherein the image sensor is a camera that is configured to simultaneously count multiple pills of the plurality of pills moving through the dispense imaging area. Any one or more of the above aspects include wherein the image sensor is a camera that is configured to recognize a color and a shape of at least one pill of the plurality of pills moving through the dispense imaging area. Any one or more of the above aspects include wherein the dispense funnel is configured to engage with an aperture of a pill container during a dispense. Any one or more of the above aspects further including: a loading station disposed adjacent the first end of the main conveyer, comprising: a container receiving space that holds a plurality of pill containers, wherein each pill container in the plurality of pill containers comprises a respective internal receiving volume; and a transfer mechanism that picks a select pill container from the plurality of pill containers and places the select pill container onto a select puck, and into a select shuttle wherein a portion of the select pill container is exposed apart from the select puck; and a labeling station that, prior to placing the select pill container onto the select puck, applies a label to an outer portion of the select pill container. Any one or more of the above aspects include wherein the label comprises the filling information for the pill order. Any one or more of the above aspects include wherein at least one of the label and an identification tag of the select puck comprises a radio frequency identification tag or barcode, and wherein at least a portion of the filling information is encoded onto the radio frequency identification tag or barcode. Any one or more of the above aspects include a plurality of radio frequency identification tag readers or barcode readers arranged at points along the length of the track, and wherein the filling information encoded onto the radio frequency identification tag or barcode is read by the plurality of radio frequency identification tag readers or barcode reader as the select puck and the select pill container moves in proximity to the plurality of radio frequency identification tag readers or barcode readers. Any one or more of the above aspects include wherein the main conveyor is arranged as a continuous loop running from the first end to the second end along a first track side and from the second end to the first end along a second track side. Any one or more of the above aspects include a plurality of shuttles that are engaged with the track of the main conveyor, the plurality of shuttles configured to move along the track, wherein the plurality of shuttles are configured to receive and carry the plurality of pucks. Any one or more of the above aspects include wherein each shuttle of the plurality of shuttles comprises: a nest comprising a slotted receiving portion, the slotted receiving portion comprising an open end and a closed end; and a magnet arranged adjacent the closed end. Any one or more of the above aspects include wherein each puck of the plurality of pucks comprises: a magnetic base ring; and a body, comprising: a base contact portion comprising a first outer diameter, the base contact portion attached to the magnetic base ring on a first side of the base contact portion; and a container receiving portion attached to the base contact portion on a second side of the base contact portion and extending a distance away from the second side of the base contact portion, the container receiving portion comprising a second outer diameter that is greater than the first outer diameter, the container receiving portion comprising a hole comprising an inner diameter, wherein the inner diameter is sized to receive a pill container. Any one or more of the above aspects include wherein the plurality of pucks are held in contact with the plurality of shuttles via magnetic attraction between the magnet and the magnetic base ring.

Exemplary aspects are directed to a method of automatically filling containers using an automated order fulfillment system, the method comprising: receiving a plurality of pill containers at a first area of the automated order fulfillment system; sorting each pill container of the plurality of pill containers based on a size of each pill container into a first lane of first pill containers and a second lane of second pill containers, wherein the first pill containers comprise a first interior volume, wherein the second pill containers comprise a second interior volume, and wherein the first interior volume is greater than the second interior volume; conveying a shuttle and puck comprising a select pill container from one of the first pill containers or the second pill containers to a labeling station; applying, by a label machine at the labeling station, a label to an outer surface of the select pill container; conveying, by a main conveyor, the shuttle and puck to a point adjacent the labeling station; transferring, via a first pick-and-place actuator, the select pill container onto the shuttle and puck, wherein the select pill container is held by the puck; conveying, by the main conveyor, the shuttle and puck comprising the select pill container to a first identification reader; reading, by the first identification reader, at least one of the label and an identification tag associated with the shuttle or puck comprising the select pill container for order information associated with a pill order for the shuttle or puck comprising the select pill container; conveying, by the main conveyor and based on the order information read by the first identification reader, the shuttle and puck comprising the select pill container to a pill dispensing system arranged along a length of the main conveyor, wherein the pill dispensing system is one of a high volume dispenser that dispenses pills at a first rate and a low volume dispenser that dispenses pills at a second rate that is lower than the first rate; and filling, by the pill dispensing system and based on the order information associated with the pill order, the select pill container with a number of pills held by the pill dispensing system.

Any one or more of the above aspects include conveying, by the main conveyor, the shuttle and puck comprising the select pill container filled with the number of pills to a capping station arranged along the length of the main conveyor; and attaching, via the capping station, a cap to the select pill container sealing the number of pills inside the select pill container. Any one or more of the above aspects include conveying, by the main conveyor after the cap is attached, the shuttle and puck comprising the select pill container filled with the number of pills, to a packaging station; transferring, via a second pick-and-place actuator, the select pill container filled with the number of pills from the shuttle and puck into the packaging station; and conveying, by the main conveyor after the select pill container filled with the number of pills is removed from the shuttle and puck, the shuttle and puck to the point adjacent the first lane of first pill containers or to the point adjacent the second lane of second pill containers. Any one or more of the above aspects include conveying, via a packaging conveyor, the select pill container filled with the number of pills removed from the shuttle and puck into a shipping container, wherein the shipping container includes an information sheet for the pill order. Any one or more of the above aspects include wherein the first identification reader is at least one of a barcode reader, a radio frequency identification tag reader, a near field communication reader, and a camera. Any one or more of the above aspects include wherein the pill dispensing system is the low volume dispenser, and wherein prior to filling the select pill container the method further comprises: transferring, by a robotic actuator, the select pill container from the shuttle and puck into the low volume dispenser, wherein the low volume dispenser is located apart a distance from the main conveyor. Any one or more of the above aspects include conveying, by the main conveyor, the shuttle and puck comprising the select pill container filled with the number of pills to a quality assurance station (or an exception handling station) arranged along the length of the main conveyor; counting the number of pills in the select pill container; determining, by a processor, whether the pill dispensing system dispensed a predetermined number of pills associated with the pill order; and verifying, by the processor, an operation of the pill dispensing system when the number of pills in the select pill container matches the predetermined number of pills associated with the pill order.

Exemplary aspects are directed to An automated pill order filling system, including: a main conveyor comprising a track arranged in a continuous loop; a plurality of shuttles configured to travel on the track along the continuous loop (which may include one or more spurs, etc.); a pill container sorting machine arranged adjacent a first point of the track, the pill container sorting machine configured to arrange a plurality of pill containers into a transfer position adjacent the main conveyor; a pick-and-place actuator arranged adjacent the transfer position, the pick-and-place actuator configured to pick a pill container from the transfer position onto a puck that is engaged with a shuttle of the plurality of shuttles; a labeling machine arranged adjacent a second point of the track, the labeling machine configured to apply a label to an outer surface of the pill container in the puck; an identification reader arranged adjacent a third point of the track, wherein the identification reader is configured to identify a pill order associated with the pill container in the puck; a first array of automatic pill dispensers arranged adjacent a fourth point of the track, wherein each automatic pill dispenser in the first array of automatic pill dispensers is configured to dispense pills at a first fill rate; a second array of automatic pill dispensers arranged adjacent the first array of automatic pill dispensers, wherein each automatic pill dispenser in the second array of automatic pill dispensers is configured to dispense pills at a second fill rate that is lower than the first fill rate and is further configured to operate independently of the first array of automatic pill dispensers; a capping station arranged adjacent a fifth point of the track, wherein the capping station is configured to attach a cap to the pill container in the puck; a quality assurance station (and/or an exception handling station to address any anomalies) arranged adjacent a sixth point of the track, wherein the quality assurance station is configured to periodically verify an operation of at least one automatic pill dispenser in the first array of automatic pill dispensers or the second array of automatic pill dispensers; a packaging station arranged adjacent a seventh point of the track, wherein the packaging station is configured to load the pill container into a shipping container; a staging area arranged adjacent an eighth point of the track, the staging area configured to return the shuttle to a pill container load area adjacent the transfer position; and a controller that moves the shuttle from the pill container load area to the first point, second point, third point, fourth point, fifth point, sixth point, seventh point, and eighth point in an order based on information in the pill order.

Any one or more of the above aspects/examples as substantially disclosed herein.

Any one or more of the aspects/examples as substantially disclosed herein optionally in combination with any one or more other aspects/examples as substantially disclosed herein.

One or means adapted to perform any one or more of the above aspects/examples as substantially disclosed herein.

Any one or more of the features disclosed herein.

Any one or more of the features as substantially disclosed herein.

Any one or more of the features as substantially disclosed herein in combination with any one or more other features as substantially disclosed herein.

Any one of the aspects/features/examples in combination with any one or more other aspects/features/examples.

Use of any one or more of the aspects or features as disclosed herein.

It is to be appreciated that any feature described herein can be claimed in combination with any other feature(s) as described herein, regardless of whether the features come from the same described example.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The terms “determine,” “calculate,” “compute,” and variations thereof, as used herein, are used interchangeably and include any type of methodology, process, mathematical operation, or technique.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. An automated pill order filling system, comprising:
a main conveyor comprising a first end and a second end, wherein the main conveyor comprises a track running from the first end to the second end;
a plurality of pucks configured to travel on the track, wherein each puck of the plurality of pucks comprises a receiving area configured to hold a respective pill container;
a first array of automatic pill dispensers disposed along a length of the track between the first end and the second end, wherein each automatic pill dispenser in the first array of automatic pill dispensers is configured to dispense a first number of pills into a first designated pill container at a first fill rate;
a second array of automatic pill dispensers disposed adjacent the first array of automatic pill dispensers, wherein each automatic pill dispenser in the second array of automatic pill dispensers is configured to dispense a second number of pills into a second designated pill container at a second fill rate and is further configured to operate independently of the first array of automatic pill dispensers, and wherein the second fill rate is lower than the first fill rate;
a processor; and
a memory coupled with and readable by the processor and storing therein instructions that, when executed by the processor, cause the processor to:
receive, via a communication network, a pill order comprising a specific number of pills that are associated with a specific pill type;
encode at least one of a specific puck and a specific pill container with filling information for the pill order, wherein the filling information includes routing instructions for the specific pill container in the automated pill order filling system;
send a first convey signal to the main conveyor, based on the routing instructions, to convey the specific puck and the specific pill container to a select automated pill dispenser selected from the first array of automatic pill dispensers or the second array of automatic pill dispensers; and
send a dispense signal to the select automated pill dispenser to dispense the specific number of pills into the specific pill container in the specific puck forming a filled pill container.

2. The automated pill order filling system of claim 1, further comprising:
a capping station disposed along the length of the track between the first end and the second end, wherein the capping station is configured to attach a cap to a pill container filled with pills, and wherein the instructions that, when executed by the processor, further cause the processor to:
send a second convey signal to the main conveyor to convey the filled pill container to the capping station; and
send an attach signal to the capping station to attach a specific cap to the filled pill container.

3. The automated pill order filling system of claim 1, wherein each automatic pill dispenser in the first array of automatic pill dispensers comprises:
a canister that is configured to receive and hold a plurality of pills;
an actuator configured to move the plurality of pills from the canister through a dispense imaging area to a dispense funnel; and
an image sensor arranged adjacent to the dispense imaging area, the image sensor configured to count each pill of the plurality of pills moving through the dispense imaging area.

4. The automated pill order filling system of claim 3, wherein the image sensor is a camera that is configured to simultaneously count multiple pills of the plurality of pills moving through the dispense imaging area.

5. The automated pill order filling system of claim 3, wherein the image sensor is a camera that is configured to recognize a color and a shape of at least one pill of the plurality of pills moving through the dispense imaging area.

6. The automated pill order filling system of claim 4, wherein the dispense funnel is configured to engage with an aperture of a pill container during a dispense.

7. The automated pill order filling system of claim 1, further comprising:
a loading station disposed adjacent the first end of the main conveyer, comprising:
a container receiving space that holds a plurality of pill containers, wherein each pill container in the plurality of pill containers comprises a respective internal receiving volume; and
a transfer mechanism that picks a select pill container from the plurality of pill containers and places the select pill container onto a select puck, wherein a portion of the select pill container is exposed apart from the select puck; and
a labeling station that, prior to placing the select pill container onto the select puck, applies a label to an outer portion of the select pill container.

8. The automated pill order filling system of claim 7, wherein the label comprises the filling information for the pill order.

9. The automated pill order filling system of claim 8, wherein at least one of the label and an identification tag of the select puck comprises a radio frequency identification tag or barcode, and wherein at least a portion of the filling information is encoded onto the radio frequency identification tag or barcode.

10. The automated pill order filling system of claim 9, further comprising:
a plurality of radio frequency identification tag readers, optical tag readers, or barcode readers arranged at points along the length of the track, and wherein the filling information encoded onto the radio frequency identification tag, optical tag, or barcode is read by the plurality of radio frequency identification tag readers, optical tag readers, or barcode readers as the select puck and the select pill container moves in proximity to the plurality of radio frequency identification tag readers, optical tag readers, or barcode readers.

11. The automated pill order filling system of claim 1, wherein the main conveyor is arranged as a continuous loop running from the first end to the second end along a first track side and from the second end to the first end along a second track side.

12. The automated pill order filling system of claim 1, further comprising:
a plurality of shuttles that are engaged with the track of the main conveyor, the plurality of shuttles configured to move along the track, wherein the plurality of shuttles are configured to receive and carry the plurality of pucks.

13. The automated pill order filling system of claim 12, wherein each shuttle of the plurality of shuttles comprises:
a nest comprising a slotted receiving portion, the slotted receiving portion comprising an open end and a closed end; and
a magnet arranged adjacent the closed end.

14. The automated pill order filling system of claim 13, wherein each puck of the plurality of pucks comprises:
a magnetic base ring; and
a body, comprising:
a base contact portion comprising a first outer diameter, the base contact portion attached to the magnetic base ring on a first side of the base contact portion; and
a container receiving portion attached to the base contact portion on a second side of the base contact portion and extending a distance away from the second side of the base contact portion, the container receiving portion comprising a second outer diameter that is greater than the first outer diameter, the container receiving portion comprising a hole comprising an inner diameter, wherein the inner diameter is sized to receive a pill container.

15. The automated pill order filling system of claim 14, wherein the plurality of pucks are held in contact with the plurality of shuttles via magnetic attraction between the magnet and the magnetic base ring.

16. A method of automatically filling containers using an automated order fulfillment system, the method comprising:
receiving a plurality of pill containers at a first area of the automated order fulfillment system;
sorting each pill container of the plurality of pill containers based on a size of each pill container into a first lane of first pill containers and a second lane of second pill containers, wherein the first pill containers comprise a first interior volume, wherein the second pill containers comprise a second interior volume, and wherein the first interior volume is greater than the second interior volume;
conveying a shuttle and puck comprising a select pill container from one of the first pill containers or the second pill containers to a labeling station;
applying, by a label machine at the labeling station, a label to an outer surface of the select pill container;
conveying, by a main conveyor, the shuttle and puck to a point adjacent the labeling station;
transferring, via a first pick-and-place actuator, the select pill container onto the shuttle and puck, wherein the select pill container is held by the puck;
conveying, by the main conveyor, the shuttle and puck comprising the select pill container to a first identification reader;
reading, by the first identification reader, at least one of the label and an identification tag associated with the shuttle or puck comprising the select pill container for order information associated with a pill order for the shuttle or puck comprising the select pill container;
conveying, by the main conveyor and based on the order information read by the first identification reader, the shuttle and puck comprising the select pill container to a pill dispensing system arranged along a length of the main conveyor, wherein the pill dispensing system is one of a high volume dispenser that dispenses pills at a first rate and a low volume dispenser that dispenses pills at a second rate that is lower than the first rate; and
filling, by the pill dispensing system and based on the order information associated with the pill order, the select pill container with a number of pills held by the pill dispensing system.

17. The method of claim 16, further comprising:
conveying, by the main conveyor, the shuttle and puck comprising the select pill container filled with the number of pills to a capping station arranged along the length of the main conveyor; and
attaching, via the capping station, a cap to the select pill container sealing the number of pills inside the select pill container.

18. The method of claim 17, further comprising:
conveying, by the main conveyor after the cap is attached, the shuttle and puck comprising the select pill container filled with the number of pills, to a packaging station;
transferring, via a second pick-and-place actuator, the select pill container filled with the number of pills from the shuttle and puck into the packaging station; and
conveying, by the main conveyor after the select pill container filled with the number of pills is removed from the shuttle and puck, the shuttle and puck to the point adjacent the first lane of first pill containers or to the point adjacent the second lane of second pill containers.

19. The method of claim 18, further comprising:
conveying, via a packaging conveyor, the select pill container filled with the number of pills removed from the shuttle and puck into a shipping container, wherein the shipping container includes an information sheet for the pill order.

20. The method of claim 16, wherein the first identification reader is at least one of a barcode reader, a radio frequency identification tag reader, a near field communication reader, and a camera.

21. The method of claim 16, wherein the pill dispensing system is the low volume dispenser, and wherein prior to filling the select pill container the method further comprises:
transferring, by a robotic actuator, the select pill container from the shuttle and puck into the low volume dispenser, wherein the low volume dispenser is located apart a distance from the main conveyor.

22. The method of claim 16, further comprising:
conveying, by the main conveyor, the shuttle and puck comprising the select pill container filled with the number of pills to a quality assurance station arranged along the length of the main conveyor;
counting the number of pills in the select pill container;
determining, by a processor, whether the pill dispensing system dispensed a predetermined number of pills associated with the pill order; and
verifying, by the processor, an operation of the pill dispensing system when the number of pills in the select pill container matches the predetermined number of pills associated with the pill order.

23. An automated pill order filling system, comprising:
a main conveyor comprising a track arranged in a continuous loop;
a plurality of shuttles configured to travel on the track along the continuous loop;
a pill container sorting machine arranged adjacent a first point of the track, the pill container sorting machine configured to arrange a plurality of pill containers into a transfer position adjacent the main conveyor;
a pick-and-place actuator arranged adjacent the transfer position, the pick-and-place actuator configured to pick a pill container from the transfer position onto a puck that is engaged with a shuttle of the plurality of shuttles;
a labeling machine arranged adjacent a second point of the track, the labeling machine configured to apply a label to an outer surface of the pill container in the puck;
an identification reader arranged adjacent a third point of the track, wherein the identification reader is configured to identify a pill order associated with the pill container in the puck;
a first array of automatic pill dispensers arranged adjacent a fourth point of the track, wherein each automatic pill dispenser in the first array of automatic pill dispensers is configured to dispense pills at a first fill rate;
a second array of automatic pill dispensers arranged adjacent the first array of automatic pill dispensers, wherein each automatic pill dispenser in the second array of automatic pill dispensers is configured to dispense pills at a second fill rate that is lower than the first fill rate and is further configured to operate independently of the first array of automatic pill dispensers;
a capping station arranged adjacent a fifth point of the track, wherein the capping station is configured to attach a cap to the pill container in the puck;
a quality assurance station arranged adjacent a sixth point of the track, wherein the quality assurance station is configured to periodically verify an operation of at least one automatic pill dispenser in the first array of automatic pill dispensers or the second array of automatic pill dispensers;
a packaging station arranged adjacent a seventh point of the track, wherein the packaging station is configured to load the pill container into a shipping container;
a staging area arranged adjacent an eighth point of the track, the staging area configured to return the shuttle to a pill container load area adjacent the transfer position; and
a controller that moves the shuttle from the pill container load area to the first point, second point, third point, fourth point, fifth point, sixth point, seventh point, and eighth point in an order based on information in the pill order.

24. The automated pill order filling system of claim 23, wherein a vision system comprising at least one camera or image sensor is configured to capture images of the pills inside the pill container.

25. The automated pill order filling system of claim 24, wherein the vision system is part of the quality assurance station.

26. The automated pill order filling system of claim 24, wherein the images of the pills inside the pill container are sent, across a communication network, to a communication device of a pharmacist such as a computer, and wherein the pharmacist compares the images of the pills inside the container to a reference image of the pill.

27. The automated pill order filling system of claim 24, wherein the images of the pills inside the pill container are sent, across a communication network, to a computer system comprising an artificial intelligence engine that is configured to automatically, and without human input, compare the images of the pills inside the container to a reference image of the pill.

28. The automated pill order filling system of claim 23, wherein the pill container comprises:
a radio frequency identification (RFID) chip integrated in the label.

29. The automated pill order filling system of claim 28, wherein the RFID chip is used to verify a prescription of the pill order associated with the pill container.

30. The automated pill order filling system of claim 23, wherein the pill container comprises:
a smart tag integrated in the label, wherein the smart tag is used to verify a prescription of the pill order associated with the pill container.
